(19) United States
(12) Patent Application Publication
(10) Pub. No.: US 2007/0258936 A1
(43) Pub. Date: Nov. 8, 2007

(54) Composition for washing keratin materials and cosmetic treatment process using said composition

(76) Inventors: Sandrine Decoster, Saint-Gratien (FR); Virginie Masse, Clichy (FR); Sandie Lesch, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/790,936

(22) Filed: Apr. 30, 2007

Related U.S. Application Data
(60) Provisional application No. 60/799,002, filed on May 10, 2006.

(30) Foreign Application Priority Data
Apr. 28, 2006 (FR) 06/03848

(51) Int. Cl. A61K 8/89; A61K 8/37

(52) U.S. Cl. 424/70.12; 424/70.31

(57) ABSTRACT

The present disclosure relates to cosmetic compositions for washing keratin materials, comprising of a microemulsion comprising: water, at least one oxyethylenated sorbitan ester, at least one oil chosen from fatty esters and amino silicones, with the proviso that said fatty esters are not oxyethylenated sorbitan esters, at least one anionic surfactant, and at least one additional surfactant chosen from amphoteric and zwitterionic surfactants. The present disclosure also relates to a cosmetic process for treating keratin materials, such as the hair or the skin, using the said composition.

This application claims benefit of U.S. Provisional Application No. 60/799,002, filed May 10, 2006, the contents of which are incorporated herein by reference. This application also claims benefit of priority under 35 U.S.C. §119 to French Patent Application No. FR 06/03848, filed Apr. 28, 2006, the contents of which are also incorporated herein by reference.

The present disclosure relates to a beneficial cosmetic composition for washing keratin materials, such as the hair and/or the skin, wherein said composition may be in the form of a microemulsion of water and at least one oil chosen from fatty esters and amino silicones.

The present disclosure also relates to a cosmetic treatment process using such a composition.

Many compositions for washing keratin materials are known.

For example, French patent application no. FR 2 804 020 describes washing compositions, such as shampoos, comprising at least one detergent surfactant and at least one oxyethylenated fatty acid ester of sorbitan, wherein the number of moles of oxyethylene is less than or equal to 10.

U.S. Pat. No. 6,153,569 describes transparent shampoo compositions that may have both good working properties (good foaming power, and ease of application) and improved conditioning power. These compositions comprise a microemulsion of an amino silicone, at least one detergent anionic surfactant, at least one foam booster, at least one pH regulator, at least one thickener and water.

European patent application no. EP 453 238 describes mild shampoo compositions whose foaming power may be improved. These compositions comprise, in an aqueous medium, from 8% to 25% by weight of a mixture of surfactants comprising:
an anionic surfactant,
an amphoteric surfactant, with the exception of betaines containing phosphorus,
an oxyalkyl or glycosyl nonionic surfactant with an HLB of at least 8.

International patent application publication no. WO 02/05758 describes a self-foaming cleansing composition whose foaming power and ease of application may be improved. This composition comprises at least one self-foaming agent combined with a mixture of surfactants, said mixture comprising at least one anionic surfactant, at least one amphoteric surfactant and, optionally, at least one nonionic surfactant. This composition may also comprise one or more cationic conditioning agents, for example cationic cellulose derivatives, cationic guar derivatives, and diallyldimethylammonium chloride derivatives and copolymers.

Canadian patent no. CA 1 077 849 describes detergent compositions and shampoos that may reduce the risks of eye irritation and whose foaming power (foam volume and stability) may be improved. These compositions comprise a particular surfactant betaine, an anionic surfactant, and a nonionic surfactant consisting of a water-soluble polyoxyethylene derivative and a hydrophobic base, wherein the mole ratio of betaine to anionic surfactant ranges from 0.9/1 to 1.1/1.

International patent application publication no. WO 03/057 185 describes cosmetic or dermatological compositions for caring for or cleansing the skin and the hair, which may combine softness and good cleansing power. These compositions are based on a synergistic combination of anionic and nonionic surfactants comprising alkylpolyglycosides, alkyl (ether) sulfates, polyethylene glycol sorbitan esters and alkyl citrate sulfosuccinates.

The compositions described in the prior art, however, can have certain insufficiencies. For example, the most efficient shampoos may cause stinging in the eyes when the diluted product runs into the region of the eyes, which occurs frequently with children. Moreover, many of these compositions may cause, in the case of individuals with sensitive skin, discomfort reactions such as redness, itching or stinging.

In addition the mild compositions proposed in the prior art may have insufficient cosmetic properties for instance, in terms of softness and, as with respect to the hair, disentangling, smoothness and lightness.

The inventors have discovered, surprisingly and unexpectedly, that by using particular microemulsions comprising certain oils (such as amino silicones and/or fatty acids) and a particular combination of surfactants, it is possible to formulate mild washing compositions that can also have excellent cosmetic properties.

For example, the compositions of the present disclosure may reduce the discomfort reactions on the skin and the scalp, and may have an excellent level of ocular tolerance. In addition, they may have very good working qualities and excellent cosmetic properties including, for example, softness and, with respect to the hair, disentangling, smoothness and lightness.

One aspect of the present disclosure is a cosmetic composition for washing keratin materials comprising a microemulsion, wherein said microemulsion comprises:
water;
at least one oxyethylenated sorbitan ester;
at least one oil chosen from fatty esters (other than oxyethylenated sorbitan esters) and amino silicones;
at least one anionic surfactant; and
at least one additional surfactant chosen from amphoteric and zwitterionic surfactants.

Another aspect of the present disclosure is a cosmetic treatment process using said composition.

Other subjects, characteristics, aspects and benefits of the present disclosure will emerge more clearly upon reading the following description and examples.

The composition according to the present disclosure is in the form of a microemulsion based on water and oil.

As used herein, the term “microemulsion,” does not mean a true emulsion, but rather a thermodynamically stable transparent solution of oil-swollen micelles. In other words, the oil is dissolved in the water in the presence of at least one surfactant. This definition is consistent with the definition of “microemulsion” in the “Dictionary of Colloid and Surface Science” by Paul Becher, published in 1990 by Marcel Dekker Inc., page 102.

In at least one embodiment of the present disclosure, the numerical mean diameter of the oil particles, measured by laser granulometry, is less than or equal to 100 nm, for example less than or equal to 50 nm, such as less than or equal to 20 nm.

The microemulsions of the present disclosure are distinguished from nanoemulsions, in that, in a nanoemulsion, the oil particles may have initial sizes of the same order of magnitude as a microemulsion, but are thermodynamically unstable and thus require a large input of energy to be prepared, and are subject to change over time.

The microemulsions of the present disclosure may have a translucent or transparent appearance. In at least one embodiment, they have a transparent appearance.

The transparency of the microemulsions of the present disclosure may be determined by measuring the transmittance at 700 nm with an absorption spectrometer in the visible region (for example the Lambda 14 spectrometer from Perkin Elmer or the UV2101 PC spectrometer from Shimadzu). These measurement may be taken on an undiluted sample of the disclosed microemulsion and compared to the transparency of a control sample, e.g., of distilled water.

The compositions according to the present disclosure may, for example, exhibit a transmittance of greater than or equal to 85%, such as greater than or equal to 90%, for instance greater than or equal to 94%. In at least one embodiment of the present disclosure, the transmittance ranges from 96% to 100%.

The microemulsions of the present disclosure may contain at least one solvent, for example, to improve the transparency of the formulation.

Non-limiting examples of such solvents include those chosen from: - C₁-C₈ lower alcohols, such as ethanol; and - glycols, such as glycerol, propylene glycol, 1,3-butylene glycol, dipropylene glycol, hexylene glycol, and polyethylene glycols containing from 4 to 16 ethylene oxide units, for example, from 8 to 12 ethylene oxide units.

The composition of the present disclosure also comprises at least one oil chosen from fatty esters (other than oxyethylenated sorbitan esters) and amino silicones.

In at least one embodiment, the at least one fatty ester is chosen from esters that are liquid at room temperature (from 20 to 30° C.) and insoluble in water at 25° C. at a concentration of greater than or equal to 0.1% by weight in water, i.e. they do not form, under these conditions, a transparent isotropic solution.

Non-limiting examples of suitable fatty esters that may be used include at least one ester of the formula RₐCOOR_{b}, wherein Rₐ is chosen from linear and branched, optionally hydroxylated, saturated and unsaturated higher carboxylic acid residues containing from 3 to 30 carbon atoms, for example, from 6 to 24 carbon atoms, and R_{b} is chosen from linear and branched, saturated and unsaturated hydrocarbon-based chains containing from 1 to 30 carbon atoms, for example, from 3 to 20 carbon atoms.

In a non-limiting embodiment, the total number of carbon atoms in the fatty ester is greater than or equal to 10. The total number of carbon atoms in the fatty ester may also be less than or equal to 100, for example less than or equal to 80, more specifically less than or equal to 50.

As examples of such esters, non-limiting mention is made of octyldodecyl behenate; isocetyl behenate; isocetyl lactate; isostearyl lactate; linoleyl lactate; oleyl lactate; isostearyl octanoate; isocetyl octanoate; decyl oleate; isocetyl isostearate; isocetyl laurate; isocetyl stearate; isodecyl octanoate; isodecyl oleate; isononyl isononanoate; isostearyl palmitate; myristyl isostearate; octyl isononanoate; 2-ethylhexyl isononanoate; octyl isostearate; octyldodecyl erucate; isopropyl palmitate, 2-ethylhexyl palmitate, 2-octyldecyl palmitate, branched alkyl myristates such as isopropyl myristate, t-butyl myristate or 2-octyldodecyl myristate, hexyl isostearate, butyl isostearate or isobutyl stearate; and 2-hexyldecyl laurate.

Additional non-limiting examples of fatty esters that may be used include esters of C₄-C₂₂ di- or tricarboxylic acids and of C₁-C₂₂ alcohols and esters of mono-, di- or tricarboxylic acids and of C₂-C₂₆ di-, tri-, tetra- or pentahydroxy alcohols.

Further non-limiting mention is made of diisopropyl sebacate; diisopropyl adipate; diisostearyl adipate; octyldodecyl stearoyl stearate; pentaerythrityl tetraisononanoate; pentaerythrityl tetraisostearate; triisopropyl citrate; triisostearyl citrate; trioctyldodecyl citrate.

In at least one embodiment, the composition of the present disclosure comprises at least one fatty ester chosen from isopropyl palmitate, 2-ethylhexyl palmitate, 2-octyldecyl palmitate, branched alkyl myristates (such as isopropyl myristate, t-butyl myristate or 2-octyldodecyl myristate), hexyl isostearate, butyl isostearate, isobutyl stearate; 2-hexyldecyl laurate, isononyl isononanoate, isostearyl neopentanoate and tridecyl neopentanoate.

In at least one embodiment, the composition of the present disclosure comprises at least one fatty ester chosen from isopropyl myristate and isostearyl neopentanoate.

The compositions according to the present disclosure may also comprise at least one oil chosen from amino silicones. As used herein, the term “amino silicone” means any silicone comprising at least one primary, secondary or tertiary amine or a quaternary ammonium group. As examples of such amino-silicones, non-limiting mention is made of:

a) polysiloxanes chosen from those of formula (A):

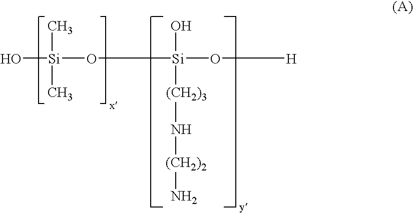

wherein x′ and y′ are integers dependent on the molecular weight of the polysiloxane, and are may be defined such that the weight-average molecular weight ranges from approximately 5,000 to 500,000;

b) 5amino silicones of formula (B):
R′ₐG₃₋ₐ-Si(OSiG₂)ₙ-(OSiG_{b}R′_{2-b})ₘ—O—SiG₃₋ₐ-R′ₐ (B)

wherein:
G, which may be identical or different, is chosen from a hydrogen atom or a phenyl group, an OH, a C₁-C₈ alkyl, for example methyl, and a C₁-C₈ alkoxy, for example methoxy;
a, which may be identical or different, is an integer ranging from 1 to 3, for example, 0;
b is 0 or 1, for example, 1,
m and n are numbers such that the sum (n+m) may range from 1 to 2,000, for example, from 50 to 150, wherein n may be a number ranging from 0 to 1,999, for example, from 49 to 149, and m may be a number ranging from 1 to 2,000, for example, from 1 to 10;
R′, which may be identical or different, is chosen from monovalent radicals of the formula —CqH2qL, wherein q is a number ranging from 2 to 8 and L is an optionally quaternized amino group chosen from the groups:
—NR″-Q-N(R″)₂
—N(R″)₂
—N⁺(R″)₃A-
—N⁺H(R″)₂A-
—N⁺H₂(R″)A-
—N(R″)-Q-N⁺R″H₂A-
—NR″-Q-N⁺(R″)₂H A-
—NR″-Q-N⁺(R″)₃A-,

wherein R″ is chosen from a hydrogen, phenyl, benzyl, or a saturated monovalent hydrocarbon-based radical, for example an alkyl radical containing from 1 to 20 carbon atoms; Q is chosen from a linear or branched group of the formula CᵣH₂ᵣ, r being an integer ranging from 2 to 6, for example, from 2 to 4; and A- is a halide ion, for example halide ions chosen from fluoride, chloride, bromide and iodide.

As an example of a group of amino silicones corresponding to this definition, non-limiting mention is made of the silicones known as “trimethylsilyl amodimethicone,” chosen from those of formula (C):

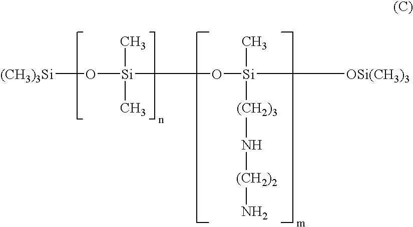

wherein n and m have the meanings given above for formula (B).

Such polymers are described, for example, in European Patent application no. EP-A-95238.

Another non-limiting example of a group of amino silicones corresponding to this definition is represented by the silicones chosen from those of formulae (D) and (E):

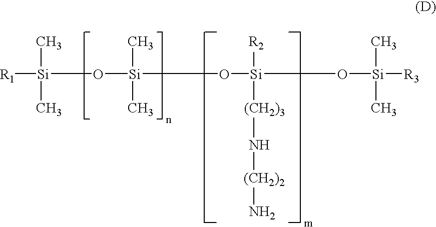

wherein:

m and n are numbers such that the sum (n+m) may range, for example from 1 to 1,000, such as from 50 to 250, more specifically from 100 to 200,

In at least one embodiment, n may be a number ranging from 0 to 999, for example from 49 to 249 and more specifically from 125 to 175, and m may be a number ranging from 1 to 1,000, for example, from 1 to 10 and more specifically from 1 to 5,

R₁, R₂ and R₃, which may be identical or different, are independently chosen from a hydroxyl and/or a C₁-C₄ alkoxy radical, wherein at least one of the radicals R₁ to R₃ is an alkoxy radical.

In a non-limiting embodiment, the alkoxy radical is a methoxy radical.

The hydroxyl/alkoxy mole ratio may range from 0.2:1 to 0.4:1, for example from 0.25:1 to 0.35:1. In at least one embodiment of the present disclosure, the hydroxy/alkoxy mole ratio is equal to 0.3:1.

The weight-average molecular mass of the silicone may range from 2,000 to 1,000,000, for example, from 3,500 to 200,000;

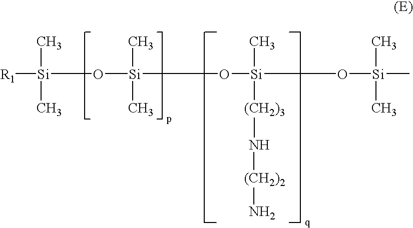

wherein:

p and q are numbers such that the sum (p+q) may range from 1 to 1,000, for example, from 50 to 350 and such as, from 150 to 250,

In at least one embodiment, p may be a number ranging from 0 to 999, for example, from 49 to 349 and such as from 159 to 239, and q may be a number ranging from 1 to 1,000, for example, from 1 to 10 and such as from 1 to 5;

R₁ and R₂, which are different, are independently chosen from a hydroxyl and C₁-C₄ alkoxy radicals, wherein at least one of the radicals R₁ and R₂ is an alkoxy radical.

In a non-limiting embodiment, the alkoxy radical is a methoxy radical.

The hydroxyl/alkoxy mole ratio can range from 1:0.8 to 1:1.1, for example, from 1:0.9 to 1:1. In a non-limiting embodiment, the hydroxyl/alkoxy mole ratio is equal to 1:0.95.

The weight-average molecular mass of the silicone may range from 2,000 to 200,000, for example, from 5,000 to 100,000 and more specifically from 10,000 to 50,000.

The weight-average molecular masses of these amino silicones are measured by gel permeation chromatography (GPC) at room temperature, as polystyrene equivalents. The columns used are p styragel columns. The eluent is THF and the flow rate is 1 ml/minute. 200 μl of a solution containing 0.5% by weight of silicone in THF are injected. Detection is performed by refractometry and UV-metry.

Commercial products that correspond to the silicones of formulae (D) and/or (E) may include in their composition at least one other amino silicones whose structure is different from those of formulae (D) and/or (E).

As an example of a commercial product containing amino silicones of structure (D), non-limiting mention is made of the product Belsil® ADM 652, sold by the company Wacker.

As an example of a commercial product containing amino silicones of formula (E), non-limiting mention is made of the product Fluid WR 1300®, sold by the company Wacker.

When these amino silicones are used, at least one embodiment of the present disclosure involves using them in the form of an oil-in-water emulsion. This oil-in-water emulsion may comprise at least one surfactant.

The surfactants may be of any nature, and may be, for example, cationic and/or nonionic.

The numerical mean size of the silicone particles in the emulsion can range from 3 nm to 500 nm.

In at least one non-limiting embodiment, microemulsions with a mean particle size ranging from 5 nm to 60 nm, for example, from 10 nm to 50 nm are used. In a further non-limiting embodiment, amino silicones of formula (E) are included in the microemulsion.

As examples of amino silicone microemulsions of comprising amino silicones of formula (E), non-limiting mention is made of the products sold under the names Finish CT 96 E® or SLM 28020® by the company Wacker.

In at least one embodiment, the amino silicone is chosen such that the contact angle with water of a hair treated with a composition containing 2% (active material) of the said silicone according to the disclosure ranges from 90° to 180°, for example, from 90° to 130°.

To measure the contact angle, the amino silicone is dissolved or dispersed in a solvent for the amino silicone or for the amino silicone emulsion (for example, hexamethyldisiloxane or water, depending on the hydrophilicity of the silicone).

In at least one embodiment, the composition containing the amino silicone(s) of formulae (D) and/or (E) is such that the contact angle with water of a hair treated with the said composition ranges from 90° to 180°, for example, from 90° to 130°.

The contact angle measurement may be based on immersing a hair in distilled water. This measurement evaluates the force exerted by the water on the hair during its immersion in distilled water and during its removal. The forces thus measured are directly related to the contact angle 0 between the water and the surface of the hair. The hair is considered hydrophilic when the angle 0 ranges from 0 to 90°, and hydrophobic when this angle ranges from 90° to 180°.

The contact angle measurement is performed with 1 g locks of natural hair that were bleached under the same conditions and then washed.

Each 1 g lock is placed in a crystallizing dish 75 mm in diameter and then uniformly covered with 5 mL of the test formulation. The lock is thus left for 15 minutes at room temperature and then rinsed with distilled water for 30 seconds. The drained lock is left to dry in the open air until it is completely dry.

For each evaluation, 10 hairs that underwent the same treatment were analyzed. Each sample, attached to a DCA (“Dynamic Contact Angle Analyzer”) precision microbalance from the company Cahn instruments, was immersed via the end in a container filled with distilled water. This DCA allows measurement of the force (f) exerted by water on the hair.

In parallel, the perimeter of the hair (p) was measured via observation under a microscope.

The mean wettability force on ten hairs and the cross section of the analysed hairs allows the contact angle of the hair on water to be determined, according to the formula:
f=p*┌lv*cos θ
wherein f is the wettability force expressed in newtons, p is the perimeter of the hair in metres, ┌lv is the liquid/vapour interface tension of the water in J/m² and θ is the contact angle.

According to the test indicated above, the product SLM 28020 from Wacker at 12% in water (i.e. 2% amino silicone) has a contact angle of 930.

According to the test indicated above, the product Belsil ADM 652 from Wacker at 2% in hexamethyldisiloxane (i.e. 2% amino silicone) has a contact angle of 111°.

Another group of amino silicones corresponding to this definition includes those of formula (F):

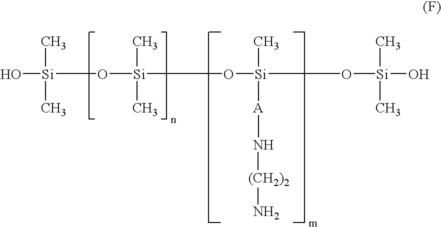

wherein:

m and n are numbers chosen such that the sum (n+m) may range from 1 to 2,000, for example, from 50 to 150. In at least one embodiment, n may be a number ranging from 0 to 1,999, for example, from 49 to 149, and m may be a number ranging from 1 to 2000, for example, from 1 to 10;

A is chosen from linear and branched alkylene radicals containing from 4 to 8 carbon atoms, for example, 4 carbon atoms. In at least one embodiment, A is a linear alkylene radical.

The weight-average molecular mass of these amino silicones may range from 2,000 to 1,000,000, for example, from 3,500 to 200,000.

The weight-average molecular masses of these amino silicones are measured by gel permeation chromatography (GPC) at room temperature, as polystyrene equivalents. The columns used are μ styragel columns. The eluent is THF and the flow rate is 1 ml/minute. 200 μl of a solution containing 0.5% by weight of silicone in THF are injected. Detection is performed by refractometry and UV-metry.

According to the present disclosure, the viscosity at 25° C. of the amino silicone may be greater than 25,000 cSt (mm²/s), for example, from 30,000 to 200,000 cSt (mm²/s) and more specifically from 30,000 to 150,000 cSt (mm²/s).

These amino silicones may have an amine number of less than or equal to 0.4 meq./g, for example, from 0.001 to 0.2 meq./g and more specifically ranging from 0.01 to 0.1 meq./g.

As used herein, the term “amine number,” means the number of amine milliequivalents per gram of compound. This number may be determined in a conventional manner, e.g., via titration methods with colored indicator or via potentiometric titration.

When these amino silicones are used, in at least one embodiment of the present disclosure, they may be used in the form of an oil-in-water emulsion. This oil-in-water emulsion may comprise at least one surfactant.

The at least one surfactant may be of any nature including, for example cationic and/or nonionic.

The numerical mean size of the silicone particles in the emulsion may range from 3 nm to 500 nm, for example, from 5 nm to 300 nm, such as from 10 nm to 275 nm and for instance, from 150 to 275 nm.

A non-limiting example of a silicone corresponding to formula (F) is DC2-8299 Cationic Emulsion from Dow Corning.

Another group of amino silicones corresponding to this definition includes those of formula (G):

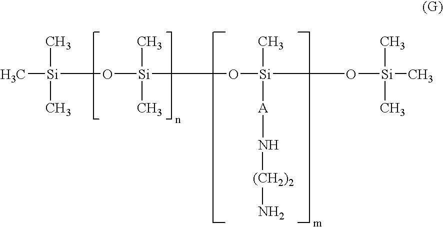

wherein:

m and n are numbers chosen such that the sum (n+m) may range from 1 to 2,000, for example, from 50 to 150 In at least one embodiment, n may be a number ranging from 0 to 1,999, for example, from 49 to 149, and m may be a number ranging from 1 to 2,00, for example, from 1 to 10;

A is chosen from linear and branched alkylene radicals containing from 4 to 8 carbon atoms, for example, 4 carbon atoms. In at least one embodiment, A is a branched alkylene radical.

The weight-average molecular mass of these amino silicones may range from 500 to 1,000,000, for example, from 1,000 to 200,000.

The weight-average molecular masses of these amino silicones are measured by gel permeation chromatography (GPC) at room temperature, as polystyrene equivalents. The columns used are μ styragel columns. The eluent is THF and the flow rate is 1 ml/minute. 200 μl of a solution containing 0.5% by weight of silicone in THF are injected. Detection is performed by refractometry and UV-metry.

According to the present disclosure, the viscosity at 25° C. of the amino silicone may be greater than 500 cSt (mm²/s), for example, from 1,000 to 200,000 cSt (mm²/s) and more specifically from 1,500 to 10,000 cSt (mm²/s).

These amino silicones may have an amine number of greater than 0.4 meq./g, for example, from 0.5 to 3 meq./g and more specifically ranging from 0.5 to 1 meq./g.

As mentioned above, as used herein the term “amine number” means the number of amine milliequivalents per gram of compound. This number may be determined in a conventional manner, e.g., via titration methods with colored indicator or via potentiometric titration.

When these amino silicones are used, in at least embodiment of the present disclosure, they may be used in the form of an oil-in-water emulsion. The oil-in-water emulsion may comprise at least one surfactant.

The at least one surfactant may be of any nature, including cationic and/or nonionic.

The numerical mean size of the silicone particles in the emulsion may range from 3 nm to 500 nm, for example, from 5 nm to 300 nm, such as from 10 nm to 275 nm and more specifically from 150 to 275 nm.

A non-limiting example of a silicone corresponding to this formula is, for example, DC2-8566 Amino Fluid from Dow Corning.

c) the amino silicones corresponding to formula (H):

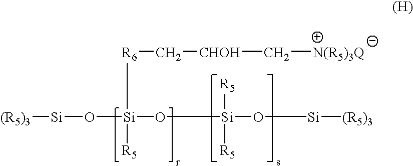

wherein:

R₅ is chosen from monovalent hydrocarbon-based radicals containing from 1 to 18 carbon atoms, for example a C₁-C₁₈ alkyl or a C₂-C₁₈ alkenyl radical, such as methyl;

R₆ is chosen from divalent hydrocarbon-based radicals, for example, C₁-C₁₈ alkylene radicals or divalent C₁-C₁₈ radicals, including, for example, C₁-C₈, alkyleneoxy radicals, and is linked to the Si via an SiC bond;

Q− is an anion, for example a halide ion such as a chloride, or an organic acid salt (e.g., an acetate, etc.);

r is chosen from a mean statistical value ranging from 2 to 20, for example, from 2 to 8;

s is chosen from a mean statistical value ranging from 20 to 200, for example from 20 to 50.

Non-limiting examples of such amino silicones are described in United States patent no. U.S. Pat. No. 4,185,087.

d) the quaternary ammonium silicones of the formula:

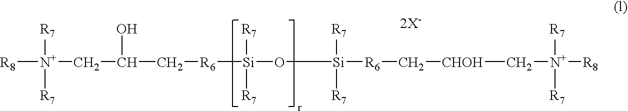

wherein:

R₇, which may be identical or different, is chosen from monovalent hydrocarbon-based radicals containing from 1 to 18 carbon atoms, for example C₁-C₁₈ alkyl radicals, C₂-C₁₈ alkenyl radicals and rings containing 5 or 6 carbon atoms, for example methyl;

R₆ is chosen from divalent hydrocarbon-based radicals, for example C₁-C₁₈ alkylene radicals or divalent C₁-C₁₈ radicals, such as a C₁-C₈, alkyleneoxy radical linked to the Si via an SiC bond;

R₈, which may be identical or different, is chosen from a hydrogen atom or a monovalent hydrocarbon-based radical containing from 1 to 18 carbon atoms, for example a C₁-C₁₈ alkyl radical, a C₂-C₁₈ alkenyl radical or a radical —R₆—NHCOR₇;

X— is an anion, for example a halide ion such as a chloride, or an organic acid salt (e.g., an acetate, etc.);

r is chosen from a mean statistical value ranging from 2 to 200, for example, from 5 to 100.

Non-limiting examples of these silicones are described in European patent application no. EP-A-0 530 974.

e) the amino silicones of formula (J):

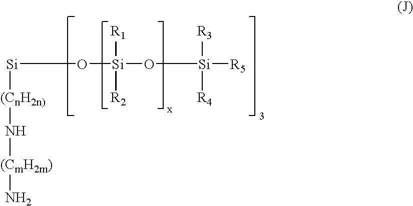

wherein:
R₁, R₂, R₃ and R₄, which may be identical or different, are independently chosen from C₁-C₄ alkyl radicals and phenyl groups,
R₅ is chosen from C₁-C₄ alkyl radicals and hydroxyl groups,
n is an integer ranging from 1 to 5,
m is an integer ranging from 1 to 5, and
x is chosen such that the amine number ranges from 0.01 to 1 meq./g.

f) the polyoxyalkylenated amino silicones of the type (XY)ᵢ, wherein i is an integer, X is a polysiloxane block and Y is a polyoxyalkylene block comprising at least one amine group, which may, for example, comprise repeating units of the formula (K):
[SiMe₂—O—(SiMe₂O)ₓSiMe₂—R—N(H)—R′—O—(C₂H₄O)ₐ—(C₃H₆O)_{b}—R′—N(H)—R—] (K)

wherein:
a is an integer greater than or equal to 1, for example, ranging from 5 to 200, more such as from 5 to 100;
b is an integer ranging from 0 to 200, for example from 4 to 200 and such as from 5 to 100;
x is an integer ranging from 1 to 10,000, for example, from 10 to 5,000;
R, which may be identical or different, is chosen from a divalent organic group that is linked to the adjacent silicon atom via a carbon-silica bond and to a nitrogen atom,
R′, which may be identical or different, is chosen from a divalent organic group that is linked to the adjacent oxygen atom via a carbon-oxygen bond and to a nitrogen atom.

R may, for example, be chosen from C₂-C₁₂ hydrocarbon-based radicals optionally comprising at least one heteroatom such as oxygen. For example R may be chosen from an ethylene, linear or branched propylene, linear or branched butylene or —CH₂CH₂CH₂OCH(OH)CH₂— radical.

R′ may, for example, be chosen from a C₂-C₁₂ hydrocarbon-based radical optionally comprising at least one heteroatom such as oxygen. For example, R′ may be chosen from a divalent alkylene radical, such as ethylene, a linear or branched propylene or a linear or branched butylene.

The siloxane blocks may be present in an amount ranging from 50 mol % to 95 mol %, for example from 70 mol % to 85 mol % relative to the total weight of the silicone.

The amine content can range from 0.02 to 0.5 meq./g, for example, from 0.05 to 0.2 meq./g of copolymer in a 30% solution in dipropylene glycol.

The weight-average molecular weight of the silicone of formula (K) may range from 5,000 to 1,000,000, for example, from 10,000 to 200,000.

As an example of a silicone of formula (K), non-limiting mention is made of the one sold under the brand name Silsoft A-843 Organosilicone Copolymer by OSI.

In at least one embodiment of the present disclosure, the amino silicones used are chosen from those of formulae (A) and (B) and mixtures thereof. In a further non-limiting embodiment, the amino silicones used are chosen from those of formulae (A) to (G), and mixtures thereof.

In at least one embodiment, the silicones used are chosen from amodimethicone and trimethylsilyl amodimethicone according to the CTFA name (2000).

In at least one embodiment, the silicone oil comprises a polydimethylsiloxane containing aminoethyl iminoisobutyl groups. As an example of such a silicone oil, non-limiting mention is made of the product sold under the name DC2-8566 Amino Fluid by the company Dow Corning.

The composition according to the present disclosure may comprise at least 0.01% by weight of oil relative to the total weight of the composition. For example, the amount of oil may range from 0.01% to 20%, such as from 0.1% to 10% by weight, more specifically from 0.2% to 5% by weight, for example from 0.5% to 3% by weight of oil, relative to the total weight of the composition.

The composition of the present disclosure further comprises at least one oxyethylenated sorbitan ester.

Examples of oxyethylenated sorbitan esters that may be used include, but are not limited to oxyethylenated derivatives of C₈-C₃₀ fatty acid monoesters and polyesters of sorbitan, containing from 1 to 50 ethylene oxide units. In at least one embodiment, oxyethylenated derivatives of C₁₂-C₂₄ fatty acid monoesters and polyesters of sorbitan, containing from 4 to 20 ethylene oxide units, are used.

These compounds are also known as polysorbates. As an example of such compounds, non-limiting mention is made of the products sold under the name Tween by the company Uniqema. Non-limiting mention is also made of: sorbitan monolaurate oxyethylenated with 4 OE, sold under the name Tween 21, sorbitan monolaurate oxyethylenated with 20 OE, sold under the name Tween 20, sorbitan monopalmitate oxyethylenated with 20 OE, sold under the name Tween 40, sorbitan monostearate oxyethylenated with 20 OE, sold under the name Tween 60, sorbitan monostearate oxyethylenated with 4 OE, sold under the name Tween 61, sorbitan tristearate oxyethylenated with 20 OE, sold under the name Tween 65, sorbitan monooleate oxyethylenated with 20 OE, sold under the name Tween 80, sorbitan monooleate oxyethylenated with 5 OE, sold under the name Tween 81, and sorbitan trioleate oxyethylenated with 20 OE, sold under the name Tween 85.

As used herein, the term “compound containing X OE” means an oxyethylenated compound comprising X oxyethylene units per molecule.

In at least one embodiment of the present disclosure, the fatty acid of the oxyethylenated sorbitan ester is a saturated fatty acid.

In a non-limiting embodiment, the sorbitan esters may be chosen, for example, from sorbitan monolaurate oxyethylenated with 4 OE, sorbitan monolaurate oxyethylenated with 20 OE, and mixtures thereof. In at least one embodiment, the sorbitan ester is sorbitan monolaurate oxyethylenated with 4 OE.

According to at least one non-limiting embodiment, the composition according to the present disclosure comprises a mixture of sorbitan monolaurate oxyethylenated with 4 OE and of sorbitan monolaurate oxyethylenated with 20 OE.

The composition according to the present disclosure may comprise a total amount of at least 0.5% by weight of oxyethylenated sorbitan ester(s) relative to the total weight of the composition. For example, the oxyethylenated sorbitan ester(s) may be present in an amount ranging from 0.5% to 20% by weight, such as from 2% to 15% by weight and more specifically from 4% to 10% by weight, relative to the total weight of the composition.

In at least one embodiment, when sorbitan monolaurate oxyethylenated with 4 OE is used, it is present in the composition of the present disclosure in an amount ranging from 2% to 10% by weight, relative to the total weight of the composition.

In at least one embodiment, when sorbitan monolaurate oxyethylenated with 20 OE is used, it is present in the composition according of the present disclosure in an amount ranging from 1% to 3% by weight, relative to the total weight of the composition.

The composition according to the present disclosure further comprises at least one anionic surfactant.

The anionic surfactants that may be used in the composition are chosen from salts, for example alkali metal salts such as sodium salts, ammonium salts, amine salts, amino alcohol salts or alkaline-earth metal salts, for example magnesium salts, of the following types: alkyl sulfates, alkyl ether sulfates, alkylamido ether sulfates, alkylarylpolyether sulfates, monoglyceride sulfates; alkyl sulfonates, alkylamide sulfonates, alkylaryl sulfonates, α-olefin sulfonates, paraffin sulfonates, alkyl sulfosuccinates, alkyl ether sulfosuccinates, alkylamide sulfosuccinates, alkyl sulfoacetates, acyl sarcosinates and acyl glutamates, wherein the alkyl and acyl groups of all these compounds contain from 6 to 24 carbon atoms, and the aryl groups are, for example, a phenyl or benzyl group.

The anionic surfactants may also be chosen from monoesters of C₆₋₂₄ alkyl and of polyglycoside-dicarboxylic acids, such as alkyl glucoside citrates, polyalkyl glycoside tartrates and polyalkyl glycoside sulfosuccinates, alkyl sulfosuccinamates, acyl isethionates and N-acyltaurates, wherein the alkyl or acyl group of all these compounds contain from 12 to 20 carbon atoms.

As additional non-limiting examples of anionic surfactants that may be used in the compositions of the present disclosure, non-limiting mention is made of acyl lactylates, wherein the acyl group contains from 8 to 20 carbon atoms.

In addition, non-limiting mention is also made of alkyl-D-galactosiduronic acids and salts thereof, and also polyoxyalkylenated (C₆₋₂₄ alkyl)ether-carboxylic acids, polyoxyalkylenated (C₆₋₂₄ alkyl)(C₆₋₂₄ aryl)ether-carboxylic acids and polyoxyalkylenated (C₆₋₂₄ alkyl)amidoether-carboxylic acids and salts thereof, including those comprising from 2 to 50 ethylene oxide units, and mixtures thereof.

In at least one embodiment of the present disclosure, the at least one anionic surfactant is chosen from alkyl sulfates and alkyl ether sulfates, and mixtures thereof, including those in the form of alkali metal, alkaline-earth metal, ammonium, amine or amino alcohol salts.

The at least one anionic surfactant may be present in an amount ranging from 0.5% to 50% by weight, for example from 4% to 20% by weight, relative to the total weight of the composition.

The composition according to the present disclosure further comprises at least one additional surfactant chosen from amphoteric and zwitterionic surfactant.

Non-limiting examples of amphoteric and/or zwitterionic surfactants that may be used in the present disclosure include aliphatic secondary or tertiary amine derivatives wherein the aliphatic group is a linear or branched chain containing from 8 to 22 carbon atoms and containing at least one anionic group, such as a carboxylate, sulfonate, sulfate, phosphate or phosphonate group. Non-limiting mention is also made of (C₈-C₂₀)alkylbetaines, sulfobetaines, (C₈-C₂₀)alkylamido(C₆-C₈)alkylbetaines or (C₈-C₂₀)alkylamido(C₆-C₈)alkylsulfobetaines.

Among the amine derivatives, non-limiting mention is made of the products sold under the name Miranol®, as described in U.S. Pat. Nos. 2,528,378 and 2,781,354 and classified in the CTFA dictionary, 3rd edition, 1982, under the names Amphocarboxyglycinate and Amphocarboxypropionate, having the respective structures (I) and (II):
Rₐ—CONHCH₂CH₂—N(R_{b})(R_{c})(CH₂COO⁻) (I)

wherein:
Rₐ is chosen from alkyl groups derived from an acid Rₐ—COOH present in hydrolysed coconut oil, a heptyl, nonyl or undecyl group,
R_{b} is a β-hydroxyethyl group, and
R_{c} is a carboxymethyl group; and
   Rₐ′—CONHCH₂CH₂—N(B)(B′) (II)

wherein:
B is —CH₂CH₂OX′,
B′ is —(CH₂)_{z}—Y′, with z=1 or 2,
X′ is chosen from a —CH₂CH₂—COOH group and a hydrogen atom,
Y′ is chosen from —COOH and a —CH₂—CHOH—SO₃H group, and
Rₐ′ is chosen from an alkyl group of an acid Rₐ′—COOH present in coconut oil or in hydrolysed linseed oil, an alkyl group, for instance a C₁₇ alkyl group and its iso form, and an unsaturated C₁₇ group.

Non-limiting examples of these compounds include those classified in the CTFA dictionary, 5th edition, 1993, under the names disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylamphodipropionate, disodium caprylamphodipropionate, lauroamphodipropionic acid, cocoamphodipropionic acid.

By way of example, non-limiting mention is made of the cocoamphodiacetate sold by the company Rhodia under the trade name Miranol® C2M concentrate.

Among the amphoteric and zwitterionic surfactants mentioned above, non-limiting mention is made of (C₈₋₂₀ alkyl)betaines, (C₈₋₂₀ alkyl)amido(C₆₋₈ alkyl)betaines, and mixtures thereof.

The amphoteric and/or zwitterionic surfactant(s) may be present in the composition in an amount ranging from 0.1% to 10% by weight, for example from 0.5% to 8% by weight relative to the total weight of the composition.

In at least one embodiment, the weight ratio of the concentration of amphoteric and/or zwitterionic surfactants(s) to the concentration of anionic surfactant(s) ranges from 0.1 to 0.5.

When the oil comprises at least one amino silicone, the weight ratio of the amount of oil to the total amount of oxyethylenated sorbitan ester(s), of anionic surfactant(s) and of amphoteric and/or zwitterionic surfactant(s)ranges from 0.01 to 0.2 and for example, from 0.02 to 0.1.

When the oil comprises at least one fatty ester, the weight ratio of the amount of oil to the total amount of oxyethylenated sorbitan ester(s), of anionic surfactant(s) and of amphoteric and/or zwitterionic surfactant(s), ranges from 0.02 to 0.4, for example, from 0.04 to 0.2.

The composition according to the present disclosure may further comprise, in addition to the oxyethylenated sorbitan ester(s), at least one other additional nonionic surfactants other than the aforementioned esters.

Non-limiting examples of additional nonionic surfactants that may be used in the compositions of the present disclosure are described in “Handbook of Surfactants” by M. R. Porter, published by Blackie & Son (Glasgow and London), 1991, pp. 116-178. Of such surfactants, non-limiting mention is made of those chosen from polyethoxylated, polypropoxylated or polyglycerolated fatty acids, (C₁-C₂₀)alkylphenols, α-diols or alcohols having a fatty chain containing, for example, 8 to 18 carbon atoms, wherein the number of ethylene oxide or propylene oxide groups optionally ranges from 2 to 50 and the number of glycerol groups ranges from 2 to 30.

Non-limiting mention may also be made of condensates of ethylene oxide and of propylene oxide with fatty alcohols; polyethoxylated fatty amides, including those having from 2 to 30 ethylene oxide units, polyglycerolated fatty amides containing on average 1 to 5, for example 1.5 to 4, glycerol groups; fatty acid esters of sucrose, fatty acid esters of polyethylene glycol, (C₆₋₂₄ alkyl)polyglycosides, N-(C₆₋₂₄ alkyl)glucamine derivatives, amine oxides such as (C₁₀-C₁₄)alkylamine oxides and N—(C₁₀-₁₄ acyl)amino-propylmorpholine oxides.

When used, the at least one additional nonionic surfactant is present in an amount ranging from 0.01% to 10% by weight, for example from 0.05% to 5% by weight, relative to the total weight of the composition.

The composition according to the present disclosure may further comprise at least one cationic surfactant.

Non-limiting examples of cationic surfactants that may be used include salts of optionally polyoxyalkylenated primary, secondary or tertiary fatty amines; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; and amine oxides of cationic nature.

When used, the at least one cationic surfactant can be present in an amount ranging from 0.01% to 10% by weight, for example from 0.05% to 5% by weight, such as from 0.3% to 3% by weight, relative to the total weight of the cosmetic composition.

The compositions according to the present disclosure may further comprise at least one cationic polymer.

As used herein, the term “cationic polymer” means any polymer containing cationic groups and/or groups that may be ionized into cationic groups.

Non-limiting examples of the cationic polymers that may be used in accordance with the present disclosure include those already known per se as improving the cosmetic properties of the hair, e.g., those described in European patent application no. EP-A-0 337 354 and in French patent application nos. FR-A-2 270 846, FR-2 383 660, FR-2 598 611, FR-2 470 596 and FR-2 519 863.

The at least one cationic polymer may be chosen, for example, from those containing units comprising primary, secondary, tertiary and/or quaternary amine groups that may either form part of the main polymer chain or may be borne by a side substituent directly attached thereto.

The at least one cationic polymer may have a weight-average molecular mass of greater than 10⁵, for example greater than 10⁶, such as from 10⁶ to 10⁸.

Among the cationic polymers that may used, non-limiting mention is made of polymers of the polyamine, polyamino amide and polyquaternary ammonium type. These are known products.

The polymers of the polyamine, polyamino amide and polyquaternary ammonium type that may be used in the composition of the present disclosure include, for example, those described in French patent nos. 2 505 348 and 2 542 997. Among these polymers, non-limiting mention is made of:

(1): homopolymers or copolymers derived from acrylic or methacrylic esters or amides containing an amine functional group, comprising at least one unit of the following formulae:

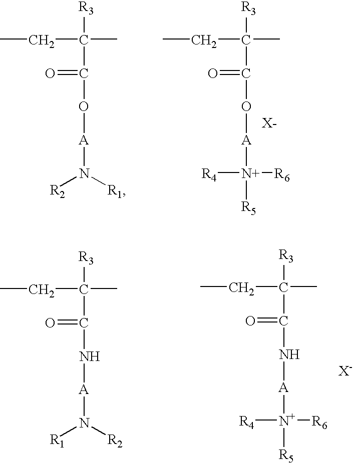

wherein:
R₁ and R₂, which may be identical or different, are independently chosen from a hydrogen atom or an alkyl group containing from 1 to 6 carbon atoms, such as a methyl or ethyl group;
R₃, which may be identical or different, is independently chosen from a hydrogen atom or a CH₃ group;
A, which may be identical or different, are independently chosen from linear and branched alkyl groups having 1 to 6 carbon atoms, such as those having 2 or-3 carbon atoms, and a hydroxyalkyl group having 1 to 4 carbon atoms;
R₄, R₅ and R₆, which may be identical or different, are independently chosen from alkyl groups containing from 1 to 18 carbon atoms and a benzyl group, including alkyl groups containing from 1 to 6 carbon atoms;
X⁻ is an anion derived from a mineral or organic acid, such as a methosulfate anion or a halide such as chloride or bromide.

The copolymers of type (1) may also contain at least one unit derived from comonomers that may be chosen from the family of acrylamides, methacrylamides, diacetone acrylamides, acrylamides and methacrylamides substituted on the nitrogen with (C₁-C₄) lower alkyl groups, groups derived from acrylic or methacrylic acids or esters thereof, from vinyllactams such as vinylpyrrolidone or vinylcaprolactam, and from vinyl esters.

Among the copolymers of type (1), non-limiting mention is made of:
copolymers of acrylamide and of dimethylaminoethyl methacrylate quaternized with dimethyl sulfate or with a dimethyl halide,
the copolymers of acrylamide and of methacryloyloxyethyltrimethylammonium chloride described, for example, in European patent application no. EP-A-080 976,
copolymers of acrylamide and of methacryloyloxyethyltrimethylammonium methosulfate,
quaternized or non-quaternized vinylpyrrolidone/dialkylaminoalkyl acrylate or methacrylate copolymers including, for example, the polymers described in French patent nos. 2 077 143 and 2 393 573,
dimethylaminoethyl methacrylate/vinylcaprolactam/vinylpyrrolidone terpolymers,
vinylpyrrolidone/methacrylamidopropyldimethylamine copolymers, and
quaternized vinylpyrrolidone/dimethylaminopropylmethacrylamide copolymers.

(2): The cellulose ether derivatives comprising quaternary ammonium groups, such as those described in French patent no.1 492 597, and including, for example the polymers sold under the names “JR” (JR 400, JR 125, JR 30M) or “LR” (LR 400, LR 30M) by the company Union Carbide Corporation. Non-limiting examples of these polymers include the polymers defined in the CTFA dictionary as hydroxyethylcellulose quaternary ammoniums that have reacted with an epoxide substituted with a trimethylammonium group.

(3): Cationic cellulose derivatives such as the copolymers of cellulose or cellulose derivatives grafted with a water-soluble quaternary ammonium monomer, described in U.S. Pat. No. 4,131,576, such as hydroxyalkylcelluloses, for instance hydroxymethyl-, hydroxyethyl- or hydroxypropylcelluloses grafted especially with a methacryloylethyltrimethylammonium, methacrylamidopropyltrimethylammonium or dimethyldiallylammonium salt.

Non-limiting examples of commercial products corresponding to this definition include the products sold under the name Celquat° L 200 and Celquat® H 100 by the company National Starch.

(4): The cationic polysaccharides described, for example, in U.S. Pat. Nos. 3,589,578 and 4,031,307, such as guar gums containing trialkylammonium cationic groups. Non-limiting mention is made of guar gums modified with a salt (e.g. chloride) of 2,3-epoxypropyltrimethylammonium.

Non-limiting mention is also made of the products sold under the trade names Jaguar® C13S, Jaguar® C15, Jaguar® C17 and Jaguar® C162 by the company Meyhall.

(5): Polymers comprising piperazinyl units and divalent alkylene or hydroxyalkylene radicals containing straight or branched chains, optionally interrupted with oxygen, sulfur or nitrogen atoms or with aromatic or heterocyclic rings, and also the oxidation and/or quaternization products of these polymers. As examples of these polymers, non-limiting mention is made of those described in French patent nos. 2 162 025 and 2 280 361.

(6): Water-soluble polyamino amides prepared, for example, by polycondensation of an acidic compound with a polyamine. These polyamino amides may be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound that is reactive with a bis-halohydrin, a bis-azetidinium, a bis-haloacyldiamine, a bis-alkyl halide, an epihalohydrin, a diepoxide or a bis-unsaturated derivative. The crosslinking agent may be used in an amount ranging from 0.025 to 0.35 mol per amine group of the polyamino amide. These polyamino amides may be alkylated or, if they contain at least one tertiary amine functional group, they may be quaternized. Such polymers are described, for example, in French patent nos. 2 252 840 and 2 368 508.

(7): Polyamino amide derivatives resulting from the condensation of polyalkylene polyamines with polycarboxylic acids followed by alkylation with difunctional agents. As examples of these derivatives, non-limiting mention is made of adipic acid/dialkylaminohydroxyalkyldialkylenetriamine polymers in which the alkyl group contains from 1 to 4 carbon atoms, such as a methyl, ethyl or propyl group, and the alkylene group contains from 1 to 4 carbon atoms, such as an ethylene group. Non-limiting examples of such derivatives include those described in French patent no.1 583 363.

Among these derivatives, non-limiting mention is made of the adipic acid/dimethylaminohydroxypropyl/diethylenetriamine polymers.

(8): Polymers obtained by the reaction of a polyalkylene polyamine containing two primary amine groups and at least one secondary amine group with a dicarboxylic acid chosen from diglycolic acid and saturated aliphatic dicarboxylic acids containing from 3 to 8 carbon atoms. The molar ratio between the polyalkylene polyamine and the dicarboxylic acid ranges from 0.8:1 to 1.4:1; the polyamino amide resulting therefrom is then reacted with epichlorohydrin in a molar ratio of epichlorohydrin relative to the secondary amine group of the polyamino amide ranging from 0.5:1 to 1.8:1. Non-limiting examples of such polymers include those described in U.S. Pat. Nos. 3,227,615 and 2,961,347.

(9): Cyclopolymers of alkyldiallylamine or of dialkyldiallylammonium, such as the homopolymers or copolymers containing, as main constituent of the chain, units chosen from those of formulae (Va) and (Vb):

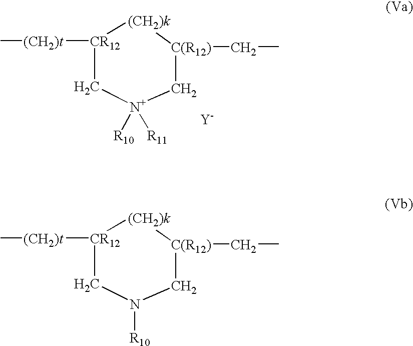

wherein;
k and t are equal to 0 or 1, the sum (k+t) being equal to 1;
R₁₂ is chosen from a hydrogen atom or a methyl radical;
R₁₀ and R₁₁, which may be the same or different, are each independently chosen from an alkyl group containing from 1 to 6 carbon atoms, a C₁₋₅ hydroxyalkyl group, a lower (C₁-C₄) amidoalkyl group, or R₁₀ and R₁₁ can, together with the nitrogen atom to which they are attached, form a heterocyclic groups such as piperidyl or morpholinyl; and
Y⁻ is an anion chosen, for example, from bromide, chloride, acetate, borate, citrate, tartrate, bisulfate, bisulfite, sulfate or phosphate.

Non-limiting examples of these polymers are described in French patent no. 2 080 759 and in its Certificate of Addition no. 2 190 406.

Alternatively, R₁₀ and R₁₁ which may be the same or different, are each independently chosen from alkyl groups containing from 1 to 4 carbon atoms.

Of the polymers defined above, non-limiting mention is made of the dimethyldiallylammonium chloride homopolymer sold under the name Merquat® 100 by the company Calgon (and its homologues of low weight-average molecular mass) and copolymers of diallyldimethylammonium chloride and of acrylamide, sold under the name Merquat® 550.

(10): The quaternary diammonium polymers containing repeating units chosen from those of formula (VI):

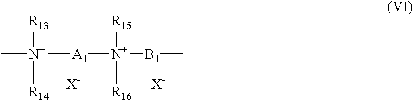

wherein:
R₁₃, R₁₄, R₁₅ and R₁₆, which may be identical or different, are each independently chosen from aliphatic, alicyclic and arylaliphatic radicals containing from 1 to 20 carbon atoms and lower hydroxyalkylaliphatic radicals, or alternatively R₁₃, R₁₄, R₁₅ and R₁₆ can, together or separately, constitute, with the nitrogen atoms to which they are attached, heterocycles optionally comprising at least one second hetero atom other than nitrogen, or alternatively R₁₃, R₁₄, R₁₅ and R₁₆ can be chosen from linear and branched C₁₋₆ alkyl radicals substituted with a nitrile, ester, acyl or amide group or a group —CO—O—R₁₇-E or —CO—NH—R₁₇-E where R₁₇ is an alkylene group and E is a quaternary ammonium group;
A₁ and B₁ are each independently chosen from polymethylene groups containing from 2 to 20 carbon atoms, wherein said groups may be linear or branched, saturated or unsaturated, and may contain, linked to or intercalated in the main chain, at least one aromatic ring or at least one oxygen or sulfur atom or sulfoxide, sulfone, disulfide, amino, alkylamino, hydroxyl, quaternary ammonium, ureido, amide or ester groups, and
X⁻ is chosen from anions derived from a mineral or organic acid;
A₁, R₁₃ and R₁₅ can form, together with the two nitrogen atoms to which they are attached, a piperazine ring; in addition, if A₁ is a linear or branched, saturated or unsaturated alkylene or hydroxyalkylene radical, B₁ can be a group:
   —(CH₂)ₙ—CO-E′-OC—(CH₂)ₙ—
   in which E′ is chosen from:

a) a glycol residue of formula: —O-Z-O—, where Z is a linear or branched hydrocarbon-based radical or a group chosen from the following formulae:
—(CH₂—CH₂—O)ₓ—CH₂—CH₂—
—[CH₂—CH(CH₃)—O]_{y}—CH₂—CH(CH₃)—

where x and y are integers ranging from 1 to 4, thereby representing a defined and unique degree of polymerization or any number ranging from 1 to 4, thus representing an average degree of polymerization;

b) a bis-secondary diamine residue such as a piperazine derivative;

c) a bis-primary diamine residue of formula —NH—Y—NH—, where Y is chosen from linear and branched hydrocarbon-based groups, or alternatively, the divalent group —CH₂—CH₂—S—S—CH₂—CH₂—; and

d) a ureylene group of formula —NH—CO—NH—.

In a non-limiting embodiment, X⁻ is an anion, such as a chloride or a bromide.

Non-limiting examples of polymers of this type are described in, for instance, French patent nos. FR 2 320 330, FR 2 270 846, FR2 316 271, FR2 336 434 and FR 2 413 907 and U.S. Pat. No. 2,273,780, U.S. Pat. No. 2,375,853, U.S. Pat. No. 2,388,614, U.S. Pat. No. 2,454,547, U.S. Pat. No. 3,206,462, U.S. Pat. No. 2,261,002, U.S. Pat. No. 2,271,378, U.S. Pat. No. 3,874,870, U.S. Pat. No. 4,001,432, U.S. Pat. No. 3,929,990, U.S. Pat. No. 3,966,904, U.S. Pat. No. 4,005,193, U.S. Pat. No. 4,025,617, U.S. Pat. No. 4,025,627, U.S. Pat. No. 4,025,653, U.S. Pat. No. 4,026,945 and U.S. Pat. No. 4,027,020.

Polymers that comprise repeating units chosen from those of formula (VII) may also be used:

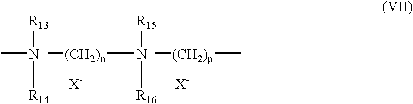

wherein R₁₃, R₁₄, R₁₅ and R₁₆, which may be identical or different, are each independently chosen from an alkyl or hydroxyalkyl group containing from 1 to 4 carbon atoms, n and p are integers ranging from 2 to 20, and X⁻ is chosen from anions derived from a mineral or organic acid.

(11): Polyquaternary ammonium polymers comprising units of formula (VIII):

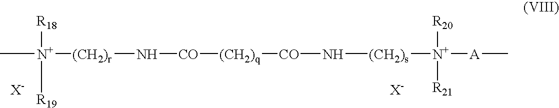

wherein:
R₁₈, R₁₉, R₂₀ and R₂₁, which may be identical or different, are each independently chosen from a hydrogen atom or a methyl, ethyl, propyl, β-hydroxyethyl, ⊕-hydroxypropyl or —CH₂CH₂(OCH₂CH₂)ₚOH radical, where p is an integer ranging from 0 to 6, with the proviso that R₁₈, R₁₉, R₂₀ and R₂₁ do not simultaneously represent a hydrogen atom,
r and s, which may be identical or different, are integers ranging from 1 to 6,
q is an integer ranging from 0 to 34,
X⁻ is chosen from anions such as halides,
A is chosen from dihalide radicals or —CH₂—CH₂—O—CH₂—CH₂—.

Non-limiting examples of such compounds are described in European patent application no EP-A-122 324.

(12): Quaternary polymers of vinylpyrrolidone and of vinylimidazole.

(13): Crosslinked methacryloyloxy(C₁₋₄)alkyltri(C₁₋₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, including, for example, methylenebisacrylamide.

Other cationic polymers which can be used in the context of the present disclosure include, for example, cationic proteins or cationic protein hydrolysates, polyalkyleneimines, such as polyethyleneimines, polymers containing vinylpyridine or vinylpyridinium units, condensates of polyamines and of epichlorohydrin, quaternary polyureylenes and cationic chitin derivatives.

Among all the cationic polymers that may be used in the context of the present disclosure, non-limiting mention is made of quaternary cellulose ether derivatives such as the products sold under the name JR 400 by the company Union Carbide Corporation, cationic cyclopolymers, such as the dimethyldiallylammonium chloride homopolymers or copolymers sold under the names Merquat® 100, Merquat® 550 and Merquat® S by the company Calgon, guar gums modified with a 2,3-epoxypropyltrimethyl-ammonium salt, and quaternary polymers of vinylpyrrolidone and of vinylimidazole.

When the at least one cationic polymer is used, it is present in the composition of the present disclosure in an amount ranging from 0.01% to 10% by weight, for example from 0.1% to 5% by weight, such as from 0.1% to 2% by weight, relative to the total weight of the composition.

The composition according to the present disclosure may further comprise at least one antidandruff agent. Non-limiting examples of antidandruff agents that may be used include compounds such as piroctone olamine, zinc pyrithione, salicylic acid and selenium disulfide, and mixtures thereof.

If used, the antidandruff agents are present in the composition of the present disclosure in an amount ranging from 0.001% to 10% by weight, for example, from 0.1% to 5% by weight, such as from 0.2% to 2% by weight, relative to the total weight of the composition.

The pH of the composition according to the present disclosure may be less than 8.5. In a non-limiting embodiment, the pH of the composition ranges from 4 to 7.

The composition according to the present disclosure may further comprise at least one adjuvant that is well known in the art, including, but not limited to hair-loss counteractants, oxidizing agents, ceramides and pseudoceramides, vitamins and provitamins including panthenol, plant, animal, mineral or synthetic oils, waxes, sunscreens, colored or uncolored mineral or organic pigments, dyes, sequestrants, plasticizers, solubilizers, acidifying agents, basifying agents, mineral or organic thickeners, antioxidants, hydroxy acids, fragrances and preserving agents.

Non-limiting examples of thickeners that may be used include cellulose derivatives, such as carboxymethylcellulose, hydroxypropylcellulose or hydroxyethylcellulose, guar gum, hydroxypropyl guar gums, scleroglucans, xanthan gum, amphiphilic polymers comprising at least one fatty chain, and fatty amides such as coconut acid monoethanolamide or coconut acid monoisopropanolamide.

A person skilled in the art will take care to select the optional additive(s) and the amount(s) thereof such that they do not harm the properties of the compositions of the present disclosure.

These adjuvants may be present in the composition according to the present disclosure in an amount ranging from 0 to 20% by weight, relative to the total weight of the composition.

The compositions according to the present disclosure may, for example, be in the form of shower gels, shampoos or compositions to be applied before or after shampooing.

In at least one non-limiting embodiment, the composition according to the present disclosure is used as a cosmetic composition for treating or caring for keratin materials, such as the hair or the skin. For example, the composition of the present disclosure may be used as a shower gel or as a shampoo. In at least one embodiment, the composition of the present disclosure is used as a shampoo.

Another aspect of the present disclosure is a cosmetic process for treating keratin materials, such as the hair, including for example a process for washing the hair. This process comprises applying an effective amount of the composition of the present disclosure to keratin materials.

In at least one non-limiting embodiment, the process of the present disclosure comprises applying to keratin materials an effective amount of the cosmetic composition and optionally in rinsing it out after an optional leave-in time.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in its respective testing measurement.

The examples that follow are intended to illustrate the present disclosure without, however, being limiting in nature.

### EXAMPLES

In the examples below, all the amounts are indicated as weight percentages of active material relative to the total weight of the-composition, unless otherwise indicated.

### Example 1

A shampoo composition in accordance with the present disclosure was prepared using the ingredients indicated in Table I below.

TABLE I

| Components | Amount |
|---|---|
| Amodimethicone (DC 2-8566 Amino Fluid from Dow | 1 |
| Corning) | |
| Hexylene glycol | 0.8 |
| PEG-5 carboxylic acid lauryl ether (Akypo RLM 45 CA from | 0.9 |
| Kao) | |
| Sodium lauryl ether sulfate containing 2.2 OE as an aqueous | 11.2 |
| solution (Texapon N702 from Cognis) | |
| Lauryl betaine (Empigen BB/LS from Huntsman) | 5.1 |
| Polyquaternium-10 (Polyquta 400 KC from KCl) | 0.4 |
| Sodium chloride | 0.2 |
| Sorbitan monolaurate oxyethylenated with 4 OE (Tween 21 | 4 |
| from Uniqema) | |
| Sorbitan monolaurate oxyethylenated with 20 OE (Tween 20 | 1.3 |
| from Uniqema) | |
| Preserving agents, fragrance | 0.5 |
| Water | qs 100 |

This composition was prepared in the following manner:

The Polyquaternium-10 was dispersed in water, with stirring, and the sodium lauryl ether sulfate was then added and dissolved, with stirring. The PEG-5 carboxylic acid lauryl ether was premixed with the preserving agents and the fragrance, and heated to 70° C., and then added.

The following was then successively added to the mixture and dissolved, with stirring: the amodimethicone, the sorbitan monolaurate oxyethylenated with 20 OE premixed with the hexylene glycol, the sorbitan monolaurate oxyethylenated with 4 OE, and finally the lauryl betaine.

The mixture was made up with water (qs 100% by weight).

### Example 2

A shampoo composition in accordance with the present disclosure was prepared using the ingredients indicated in Table II below.

TABLE II

| Component | Amount |
|---|---|
| Amodimethicone (DC 2-8566 Amino Fluid from Dow | 1 |
| Corning) | |
| Hexylene glycol | 1.6 |
| PEG-5 carboxylic acid lauryl ether (Akypo RLM 45 CA from | 0.9 |
| Kao) | |
| Sodium lauryl ether sulfate containing 2.2 OE as an aqueous | 11.2 |
| solution (Texapon N702 from Cognis) | |
| Lauryl betaine (Empigen BB/LS from Huntsman) | 5.1 |
| Polyquaternium-10 (Polyquta 400 KC from KCl) | 0.4 |
| Sodium chloride | 0.2 |
| Sorbitan monolaurate oxyethylenated with 4 OE (Tween 21 | 8 |
| from Uniqema) | |
| Sorbitan monolaurate oxyethylenated with 20 OE (Tween 20 | 2.7 |
| from Uniqema) | |
| Preserving agents, fragrance | 0.5 |
| Water | qs 100 |

This composition was prepared in a manner identical to that for the composition of Example 1.

### Example 3

A shampoo composition in accordance with the present disclosure was prepared using the ingredients indicated in Table III below.

TABLE III

| Components | Amount |
|---|---|
| Sodium lauryl ether sulfate containing 2.2 OE as an aqueous | 12.6 |
| solution (Texapon N702 from Cognis) | |
| Lauryl betaine (Empigen BB/LS from Huntsman) | 2.1 |
| Sorbitan monolaurate oxyethylenated with 4 OE (Tween 21 | 6 |
| from Uniqema) | |
| Sorbitan monolaurate oxyethylenated with 20 OE (Tween 20 | 2 |
| from Uniqema) | |
| Isostearyl neopentanoate (Ceraphyl 375 from ISP) | 1 |
| Fragrance | 0.5 |
| Mixture of methyl, butyl, ethyl, propyl and isobutyl p- | 0.5 |
| hydroxybenzoates (Nipastat from Nipa) | |
| Hexylene glycol | 1 |
| Coconut acid monoethanolamide (Comperlan from Cognis) | 1.5 |
| Polyquaternium-10 (Polyquta 400 KC from KCl) | 0.3 |
| Salicylic acid powder | 0.2 |
| Benzoic acid | 0.2 |
| Sodium chloride | 0.07 |
| Water | qs 100 |

This composition was prepared in a manner identical to that for the composition of Example 1.

### Example 4

A shampoo composition in accordance with the present disclosure was prepared using the ingredients indicated in Table IV below.

TABLE IV

| Components | Amount |
|---|---|
| Sodium lauryl ether sulfate containing 2.2 OE as an aqueous | 14.2 |
| solution (Texapon N702 from Cognis) | |
| Lauryl betaine (Empigen BB/LS from Huntsman) | 1.9 |
| Sorbitan monolaurate oxyethylenated with 4 OE (Tween 21 | 6 |
| from Uniqema) | |
| Sorbitan monolaurate oxyethylenated with 20 OE (Tween 20 | 2 |
| from Uniqema) | |
| Isopropyl myristate (Isopropyl myristate (IPM) from Cognis) | 2 |
| PEG-5 carboxylic acid lauryl ether (Akypo RLM 45 CA from | 1.8 |
| Kao) | |
| Propylene glycol PEG-55 oleate (Antil 141 Liquid from | 0.4 |
| Goldschmidt) | |
| Ceteareth-60 myristyl glycol (Elfacos GT 182 S from Akzo | 0.5 |
| Nobel) | |
| Fragrance | 0.5 |
| Mixture of methyl, butyl, ethyl, propyl and isobutyl | 0.5 |
| p-hydroxybenzoates (Nipastat from Nipa) | |
| Propylene glycol | 0.5 |
| Hexylene glycol | 0.5 |
| Polyquaternium-10 (Polyquta 400 KC from KCl) | 0.3 |
| Salicylic acid powder | 0.2 |
| Benzoic acid | 0.2 |
| Sodium chloride | 0.07 |
| Water | qs 100 |

This composition was prepared in a manner identical to that for the composition of Example 1.

### Example 5

A shampoo composition in accordance with the present disclosure was prepared using the ingredients indicated in Table V below.

TABLE V

| Components | Amount |
|---|---|
| Sodium lauryl ether sulfate containing 2.2 OE as an aqueous | 11.2 |
| solution (Texapon N702 from Cognis) | |
| Lauryl betaine (Empigen BB/LS from Huntsman) | 5.1 |
| Sorbitan monolaurate oxyethylenated with 4 OE (Tween 21 | 6 |
| from Uniqema) | |
| Sorbitan monolaurate oxyethylenated with 20 OE (Tween 20 | 2 |
| from Uniqema) | |
| Amodimethicone (DC 2-8566 Amino Fluid from Dow | 1 |
| Corning) | |
| PEG-5 carboxylic acid lauryl ether (Akypo RLM 45 CA from | 0.9 |
| Kao) | |
| Fragrance | 0.5 |
| Mixture of methyl, butyl, ethyl, propyl and isobutyl p- | 0.5 |
| hydroxybenzoates (Nipastat from Nipa) | |
| Hexylene glycol | 1.2 |
| Polyquaternium-10 (Polyquta 400 KC from KCl) | 0.4 |
| Salicylic acid powder | 0.2 |
| Sodium benzoate | 0.5 |
| Sodium chloride | 0.2 |
| Water | qs 100 |

This composition was prepared in a manner identical to that for the composition of Example 1.

### Example 6

A shampoo composition in accordance with the present disclosure was prepared using the ingredients indicated in Table VI below.

TABLE VI

| Components | Amount |
|---|---|
| Amodimethicone (DC 2-8566 Amino Fluid from Dow | 1 |
| Corning) | |
| Hexylene glycol | 1.2 |
| PEG-5 carboxylic acid lauryl ether (Akypo RLM 45 CA from | 0.9 |
| Kao) | |
| Sodium lauryl ether sulfate containing 2.2 OE as an aqueous | 11.2 |
| solution (Texapon N702 from Cognis) | |
| Lauryl betaine (Empigen BB/LS from Huntsman) | 5.1 |
| Polyquaternium-10 (Polyquta 400 KC from KCl) | 0.4 |
| Propylene glycol PEG-55 oleate (Antil 141 Liquid from | 0.3 |
| Goldschmidt) | |
| Sorbitan monolaurate oxyethylenated with 4 OE (Tween 21 | 6 |
| from Uniqema) | |
| Preserving agents, fragrance | 0.5 |
| Water | qs 100 |

This composition was prepared in a manner identical to that for the composition of Example 1.

The compositions prepared according to Examples 1 to 6 above exhibited excellent tolerance with respect to the scalp.

In addition, these compositions were extremely mild and exhibited excellent ocular tolerance.

Finally, these compositions exhibited noteworthy cosmetic properties in terms of softness, disentangling, smoothness of the hair, styling, volume and lightness.

What is claimed is:

1. A cosmetic composition for washing keratin materials, comprising a microemulsion comprising:
water,
at least one oxyethylenated sorbitan ester,
at least one oil chosen from fatty esters and amino silicones, with the proviso that said fatty esters are not oxyethylenated sorbitan esters,
at least one anionic surfactant, and
at least one additional surfactant chosen from amphoteric and zwitterionic surfactants.

2. The cosmetic composition of claim 1, wherein said composition comprises at least one fatty ester of the formula RₐCOOR_{b}, wherein Rₐ is chosen from linear and branched, optionally hydroxylated, saturated and unsaturated higher carboxylic acid residues containing from 3 to 30 carbon atoms, and R_{b} is chosen from linear and branched, saturated and unsaturated hydrocarbon-based chains containing from 1 to 30 carbon atoms.

3. The cosmetic composition of claim 2, wherein the total number of carbon atoms in said at least one fatty ester is greater than or equal to 10.

4. The cosmetic composition of claim 2, wherein the total number of carbon atoms in said at least one fatty ester is less than or equal to 100.

5. The cosmetic composition of claim 1, wherein said composition comprises at least one fatty ester chosen from esters of C₄-C₂₂ di- or tricarboxylic acids and of C₁-C₂₂ alcohols, and esters of mono-, di- or tricarboxylic acids and of C₂-C₂₆ di-, tri-, tetra- or pentahydroxy alcohols.

6. The cosmetic composition of claim 1, wherein said composition comprises at least one fatty ester chosen from isopropyl palmitate, 2-ethylhexyl palmitate, 2-octyldecyl palmitate, branched alkyl myristates (such as isopropyl myristate, t-butyl myristate or 2-octyldodecyl myristate), hexyl isostearate, butyl isostearate, isobutyl stearate; 2-hexyldecyl laurate, isononyl isononanoate, isostearyl neopentanoate and tridecyl neopentanoate.

7. The cosmetic composition of claim 6, wherein said composition comprises at least one fatty ester chosen from isopropyl myristate and isostearyl neopentanoate.

8. The cosmetic composition of claim 1, wherein said composition comprises at least one amino silicone chosen from:
a) polysiloxanes chosen from those of formula (A): 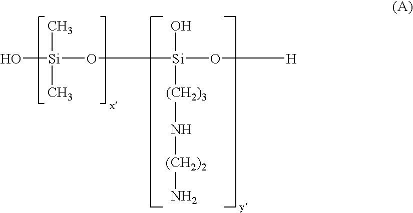
wherein x′ and y′ are integers chosen such that the weight-average molecular weight of said polysiloxane ranges from 5,000 to 500,000;
b) amino silicones chosen from those of formula (B):
R′ₐG₃₋ₐ-Si(OSiG₂)ₙ—(OSiG_{b}R′_{2-b})ₘ—O—SiG₃₋ₐ-R′ₐ (B)
wherein:
G, which may be identical or different, is independently chosen from a hydrogen atom and a phenyl, OH, C₁-C₈ alkyl and C₁-C₈ alkoxy groups,
a, which may be identical or different, is an integer ranging from 0 to 3,
b is 0 or 1,
m and n are numbers chosen such that the sum (n+m) ranges from 1 to 2,000;
R′, which may be identical or different, is a monovalent radical of formula —CqH2qL wherein q is a number ranging from 2 to 8 and L is an optionally quaternized amino group chosen from the groups:
—NR″-Q-N(R″)₂,
—N(R″)₂,
—N⁺(R″)₃A-,
—N⁺H(R″)₂A-,
—N⁺H₂(R″)A-,
—N(R″)-Q-N⁺R″H₂A-,
—NR″-Q-N⁺(R″)₂H A-,
—NR″-Q-N⁺(R″)₃A-,
wherein R″ is chosen from a hydrogen atom and a phenyl, a benzyl, or a saturated monovalent hydrocarbon-based radical; Q is chosen from linear and branched groups of the formula CᵣH₂ᵣ, r being an integer ranging from 2 to 6; and A- is a halide ion;
c) the amino silicones chosen from those of formula (H): 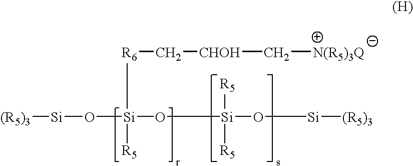
wherein:
R₅ is chosen from monovalent hydrocarbon-based radicals containing from 1 to 18 carbon atoms;
R₆ represents divalent hydrocarbon-based radicals linked to the Si via an SiC bond;
Q⁻ is an anion chosen from halide ions and organic acid salts;
r is chosen from a mean statistical value ranging from 2 to 20;
s is chosen from a mean statistical value ranging from 20 to 200; d) the quaternary ammonium silicones chosen from those of formula (I): 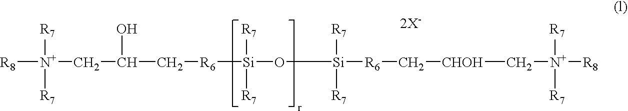
wherein:
R₇, which may be the same or different, is independently chosen from monovalent hydrocarbon-based radicals containing from 1 to 18 carbon atoms;
R₆ is chosen from divalent hydrocarbon-based radicals linked to the Si via an SiC bond;
R₈, which may be the same or different, is independently chosen from a hydrogen atom and monovalent hydrocarbon-based radicals containing from 1 to 18 carbon atoms;
X— is an anion chosen from halide ions and organic acid salts;
r is chosen from a mean statistical value ranging from 2 to 200;
e) the amino silicones chosen from those of formula (J): 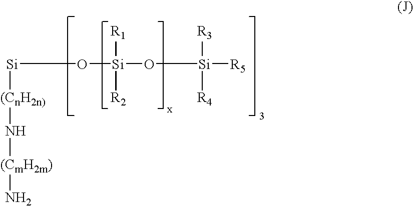
wherein:
R₁, R₂, R₃ and R₄, which may be the same or different, are each independently chosen from C₁-C₄ alkyl radicals and phenyl groups,
R₅ is chosen from C₁-C₄ alkyl radicals and hydroxyl groups,
n is an integer ranging from 1 to 5,
m is an integer ranging from 1 to 5, and
x is chosen such that the amine number of the amino silicone of formula (J) ranges from 0.01 to 1 meq./g;
f) the polyoxyalkylenated amino silicones chosen from those of the type (XY)ᵢ, wherein i is an integer, X is a polysiloxane block and Y is a polyoxyalkylene block comprising at least one amine group that may comprise repeating units chosen from those of formula (K):
[SiMe₂—O—(SiMe₂O)ₓSiMe₂—R—N(H)—R—O−(C₂H₄)ₐ—(C₃H₆)_{b}—R—N(H)—R—] (K)
wherein:
a is an integer greater than or equal to 1;
b is an integer ranging from 0 to 200;
x is an integer ranging from 1 to 10,000;
R, which may be the same or different, is independently chosen from divalent organic groups that are linked to the adjacent silicon atom via a carbon-silica bond and to a nitrogen atom,
R′, which may be the same or different, is independently chosen from divalent organic groups that are linked to the adjacent oxygen atom via a carbon-oxygen bond and to a nitrogen atom.

9. The cosmetic composition of claim 8, wherein in said formula (B), n is a number ranging from 49 to 149 and m is a number ranging from 1 to 10.

10. The cosmetic composition of claim 8, wherein in said formula (J), a is an integer ranging from 5 to 100, b is an integer ranging from 4 to 200, and x is an integer ranging from 10 to 5,000.

11. The cosmetic composition of claim 8, wherein said composition comprises at least one amino silicone chosen from those of formulae (A) and (B).

12. The cosmetic composition of claims 8, wherein the at least one amino silicone oil comprises a polydimethylsiloxane containing aminoethyl iminoisobutyl groups.

13. The cosmetic composition of claim 1, wherein the at least one oil is present in an amount of greater than or equal to 0.01% by weight relative to the total weight of the composition.

14. The cosmetic composition of claim 13, wherein said at least one oil is present in the composition in an amount ranging from 0.01% to 20% by weight, relative to the total weight of the composition.

15. The cosmetic composition of claim 14, wherein said at least one oil is present in the composition in an amount ranging from 0.1% to 10% by weight, relative to the total weight of the composition.

16. The cosmetic composition of claim 1, wherein said composition comprises at least one oxyethylenated sorbitan ester chosen from oxyethylenated derivatives of C₈-C₃₀ fatty acid monoesters and polyesters of sorbitan, containing from 1 to 50 ethylene oxide units.

17. The cosmetic composition of claim 16, wherein said composition comprises at least one oxyethylenated derivative of C₁₂-C₂₄ fatty acid monoesters and polyesters of sorbitan, containing from 4 to 20 ethylene oxide units.

18. The cosmetic composition of claim 17, wherein said composition comprises sorbitan monolaurate oxyethylenated with 4 OE.

19. The cosmetic composition of claim 16, wherein said composition comprises a mixture of sorbitan monolaurate oxyethylenated with 4 OE and of sorbitan monolaurate oxyethylenated with 20 OE.

20. The cosmetic composition of claim 1, wherein the at least one oxyethylenated sorbitan ester is present in a total amount greater than or equal to 0.5% by weight, relative to the total weight of the composition.

21. The cosmetic composition of claim 20, wherein said at least one oxyethylenated sorbitan ester is present in an amount ranging from 0.5% to 20% by weight, relative to the total weight of the composition.

22. The cosmetic composition of claim 21, wherein said at least one oxyethylenated sorbitan ester is present in an amount ranging from 4% to 10% by weight, relative to the total weight of the composition.

23. The cosmetic composition of claim 1, wherein said composition comprises at least one anionic surfactant chosen from salts of the following types: alkyl sulfates, alkyl ether sulfates, alkylamido ether sulfates, alkylarylpolyether sulfates, monoglyceride sulfates; alkyl sulfonates, alkylamide sulfonates, alkylaryl sulfonates, α-olefin sulfonates, paraffin sulfonates, alkyl sulfosuccinates, alkyl ether sulfosuccinates, alkylamide sulfosuccinates, alkyl sulfoacetates, acyl sarcosinates and acyl glutamates, wherein the alkyl and acyl groups of these compounds contain from 6 to 24 carbon atoms.

24. The cosmetic composition of claim 1, wherein said composition comprises at least one anionic surfactant chosen from C₆-C₂₄ alkyl monoesters of polyglycosidedicarboxylic acids.

25. The cosmetic composition of claim 24, wherein said at least one C₆-C₂₄ alkyl monoester of polyglycosidedicarboxylic acid is chosen from alkyl glucoside citrates, alkyl polyglycoside tartrates, alkyl polyglycoside sulfosuccinates, alkyl sulfosuccinamates, acyl isethionates and N-acyltaurates, wherein the alkyl or acyl group of all these compounds contains from 12 to 20 carbon atoms.

26. The cosmetic composition of claim 1, wherein said composition comprises at least one anionic surfactant chosen from acyl lactylates in which the acyl group contains from 8 to 20 carbon atoms, alkyl-D-galactoside uronic acids, polyoxyalkylenated (C₆-C₂₄ alkyl) ether carboxylic acids, polyoxyalkylenated (C₆-C₂₄ alkyl)(C₆-C₂₄ aryl)ether carboxylic acids, polyoxyalkylenated (C₆-C₂₄ alkyl)amidoether carboxylic acids, and the salts thereof.

27. The cosmetic composition of claim 20, wherein said composition comprises at least one anionic surfactant chosen from alkyl sulfates and alkyl ether sulfates.

28. The cosmetic composition of claim 27, wherein said at least one anionic surfactant is in the form of alkali metal or alkaline-earth metal, ammonium, amine or amino alcohol salts.

29. The cosmetic composition of claim 1, wherein said at least one anionic surfactant is present in an amount ranging from 0.5% to 50% by weight, relative to the total weight of the composition.

30. The composition of claim 29, wherein said at least one anionic surfactant is present in an amount ranging from 4% to 20% by weight, relative to the total weight of the composition.

31. The cosmetic composition of claim 1, wherein said at least one additional surfactant chosen from amphoteric and zwitterionic surfactants is chosen from:
secondary and tertiary aliphatic amine derivatives, wherein the aliphatic group is a linear or branched chain containing from 8 to 22 carbon atoms and contains at least one anionic group, and
(C₈-C₂₀)alkylbetaines, sulfobetaines, (C₈-C₂₀ alkyl)amido(C₆-C₈ alkyl)betaines or (C₈-C₂₀ alkyl)amido(C₆-C₈ alkyl)sulfobetaines.

32. The cosmetic composition of claim 31, wherein said at least one anionic group is chosen from carboxylate, sulfonate, sulfate, phosphate and phosphonate groups.

33. The cosmetic composition of claim 1, wherein said at least one additional surfactant chosen from amphoteric and zwitterionic surfactants is present in an amount ranging from 0.1% to 10% by weight, relative to the total weight of the composition.

34. The cosmetic composition of claim 33, wherein said at least one additional surfactant chosen from amphoteric and zwitterionic surfactants is present in an amount ranging from 0.5% to 8% by weight, relative to the total weight of the composition.

35. The cosmetic composition of claim 1, wherein said at least one oil comprises at least one amino silicone, and the weight ratio of the amount of said at least one oil to the total amount of said at least one oxyethylenated sorbitan ester, at least one anionic surfactant and at least one additional surfactant chosen from amphoteric and zwitterionic surfactants, ranges from 0.01 to 0.2.

36. The cosmetic composition of claim 35, wherein the weight ratio of the amount of said at least one oil to the total amount of said at least one oxyethylenated sorbitan ester, at least one anionic surfactant and at least one additional surfactant chosen from amphoteric and zwitterionic surfactants, ranges from 0.02 to 0.1.

37. The cosmetic composition of claim 1, wherein said at least one oil comprises at least one fatty ester and the weight ratio of the amount of said at least one oil to the total amount of said at least one oxyethylenated sorbitan ester, at least one anionic surfactant and at least one additional surfactant chosen from amphoteric and zwitterionic surfactants, ranges from 0.02 to 0.4.

38. The cosmetic composition of claim 37, wherein said at least one oil comprises at least one fatty ester and the weight ratio of the amount of said at least one oil to the total amount of said at least one oxyethylenated sorbitan ester, at least one anionic surfactant and at least one additional surfactant chosen from amphoteric and zwitterionic surfactants, ranges from 0.04 to 0.2.

39. The cosmetic composition of claim 1, wherein the weight ratio of the concentration of the at least one additional surfactant chosen from amphoteric and zwitterionic surfactants to the concentration of said at least one anionic surfactant ranges from 0.1 to 0.5.

40. The cosmetic composition of claim 1, further comprising at least one cationic surfactant.

41. The cosmetic composition of claim 40, wherein the at least one cationic surfactant is present in an amount ranging from 0.01% to 10% by weight, relative to the total weight of the composition.

42. The cosmetic composition of claim 41, wherein the at least one cationic surfactant is present in an amount ranging from 0.05% to 5% by weight, relative to the total weight of the composition.

43. The cosmetic composition of claim 1 further comprising at least one cationic polymer.

44. The cosmetic composition of claim 43, wherein said at least one cationic polymer is present in an amount ranging from 0.01% to 10% by weight, relative to the total weight of the composition.

45. The cosmetic composition of claim 1, further comprising at least one antidandruff agent.

46. The cosmetic composition of claim 45, wherein said at least one antidandruff agent is chosen from piroctone olamine, zinc pyrithione, salicylic acid, and selenium disulfide.

47. The cosmetic composition of claim 45, wherein said antidandruff agent is present in an amount ranging from 0.001% to 10% by weight, relative to the total weight of the composition.

48. The cosmetic composition of claim 47, wherein said antidandruff agent is present in an amount ranging from 0.1% to 5% by weight, relative to the total weight of the composition.

49. The cosmetic composition of claim 1, further comprising at least one adjuvant chosen from hair-loss counteractants; oxidizing agents; ceramides and pseudoceramides; vitamins and provitamins; plant, animal, mineral and synthetic oils; waxes; sunscreens; colored and uncolored mineral and organic pigments; dyes; sequestrants; plasticizers; solubilizers; acidifying agents; basifying agents; mineral and organic thickeners; antioxidants; hydroxy acids; fragrances; and preserving agents.

50. A process for cosmetically treating keratin materials, comprising:
applying to the keratin materials a cosmetic composition comprising a microemulsion comprising:
water,
at least one oxyethylenated sorbitan ester,
at least one oil chosen from fatty esters and amino silicones, with the proviso that said fatty esters are not oxyethylenated sorbitan esters,
at least one anionic surfactant, and
at least one additional surfactant chosen from amphoteric and zwitterionic surfactants.

51. The process of claim 50, wherein said cosmetic composition is a shampoo.
